(19) United States
(12) Patent Application Publication
(10) Pub. No.: US 2013/0192601 A1
(43) Pub. Date: Aug. 1, 2013

(54) Flow-Inflating Mask Interface for Noninvasive Positive Pressure Ventilation

(71) Applicants: Uwe Reischl, Boise, ID (US); Lonny Ashworth, Boise, ID (US); Lutana Haan, Boise, ID (US)

(72) Inventors: Uwe Reischl, Boise, ID (US); Lonny Ashworth, Boise, ID (US); Lutana Haan, Boise, ID (US)

(73) Assignee: Boise State University, Boise, ID (US)

(21) Appl. No.: 13/361,484

(22) Filed: Jan. 30, 2012

(51) Int. Cl. A61M 16/06

(52) U.S. Cl. 128/205.25

(57) ABSTRACT

A flow-inflating respiratory face mask is disclosed that includes a thin, pliant skirt that surrounds the base of the face mask. The skirt inflates automatically when the face mask receives external pressurized air. The skirt is compliant, soft, and adapts easily to the topography of the face which produces a good fit and adds to the comfort of the patient.

### BACKGROUND

The present disclosure relates, in general, to equipment used in the treatment of respiratory ailments and, more particularly, to an improved noninvasive positive-pressure ventilation (NPPV) apparatus for delivering a fluid pressure to a patient in order to maintain the patient's airway open, provide ventilatory support and deliver oxygen to such a patient.

Positive airway pressure is used in the treatment of obstructive sleep apnea (OSA) and ventilatory failure. Fluid pressure of air and/or oxygen is applied to a subject patient through the use of a respiratory face mask. Continuous Positive Airway Pressure (CPAP) is air pressure that supports the airway of the patient by acting like an artificial splint wherein there is the same level of pressure on inspiration as on expiration. For the treatment of OSA, CPAP is often used to prevent the airway from collapsing during sleep. NPPV may also be used in another way to deliver pressure wherein there is an increase in pressure on inspiration and a lower pressure during exhalation. NPPV can be used for non-invasive ventilation in that it provides a potential increase in breath volumes and end expiratory pressure to improve the patient's breathing. NPPV is a common treatment for patients suffering from ventilatory muscle fatigue, weakened ventilatory muscles, increased demand on ventilatory muscles and other disorders necessitating ventilatory support.

As can be appreciated, the fit of the face mask is crucial in that it provides the interface between the machine used to deliver CPAP or NPPV and the person. Often, the interface mask must be fairly tight in order to achieve the necessary seal to allow various air pressure and flow rates. Current face mask designs, however, generally do not provide a good fit on the face of the patient and therefore often fail to generate adequate air pressure and flow rate conditions. This problem is exacerbated when medical tubing, such as feeding and suction tubes are required to pass into or out of the patient's mouth and nose, thereby creating regions about the mask where the tubing breaks the seal. Moreover, prior designs cause discomfort for patients and can sometimes result in the development of sores on the face of the patient when used for extended periods of time.

### SUMMARY OF THE INVENTION

The present disclosure relates, in general, to equipment used in the treatment of respiratory ailments and, more particularly, to an improved positive-pressure ventilation apparatus for delivering a fluid pressure to a patient in order to maintain the patient's airway open, provide ventilatory support and deliver oxygen to such patient.

In some aspects of the disclosure, a flow-inflating mask for delivering a positive pressure fluid to a patient is disclosed. The mask may include a rigid base dimensioned to cover at least a nose and a mouth of the patient and defining an interior configured to receive the nose and mouth. The mask may further include a flow-inflating skirt coupled to the rigid base and having a distal portion that inverts towards the interior of the base and thereby defines a cavity, the cavity being configured to receive pressurized air and inflate the flow-inflating skirt.

In some aspects of the disclosure, a method for delivering a positive pressure fluid to a patient is disclosed. The method may include providing a flow of positive pressure fluid to a rigid face mask base defining an interior dimensioned to cover the nose and mouth of the patient. The method may further include inflating a flow-inflating skirt with the flow of positive pressure fluid, the flow-inflating skirt being coupled to the rigid face mask base and having a distal portion that inverts towards the interior of the face mask base to define a cavity.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modifications, alterations, combinations, and equivalents in form and function, as will occur to those skilled in the art and having the benefit of this disclosure.

FIG. 1 illustrates a perspective view of an exemplary flow-inflating mask as seated on the face of a subject patient, according to one or more embodiments.

FIG. 2a illustrates a bottom view of the exemplary flow-inflating mask, according to one or more embodiments.

FIG. 2b illustrates a top view of the exemplary flow-inflating mask, according to one or more embodiments.

FIG. 3 illustrates a cross-sectional view of the flow-inflating mask as installed adjacent to the skin of the subject patient, according to one or more embodiments.

### DETAILED DESCRIPTION

The present disclosure relates, in general, to equipment used in the treatment of respiratory ailments and, more particularly, to an improved positive-pressure ventilation apparatus for delivering a fluid pressure to a patient in order to maintain the patient's airway open, provide positive pressure ventilation, and deliver oxygen to such a patient.

The present invention provides a flow-inflating face mask that includes an inflatable “skirt” or shroud that surrounds the rigid base portion of the face mask. The skirt is designed to inflate automatically when the face mask receives a supply of positive pressure air or other fluid. The skirt is generally pliable, soft, and adapts easily to the topography of the face which produces a good fit and adds to the comfort of the patient. Because of the pliant nature of the skirt, the flow-inflating face mask can be adapted to most face mask designs. In operation, the skirt provides a more efficient and more comfortable seal between the mask and the face than conventional face mask products. For example, the flow-inflating skirt imposes less pressure on the skin tissue than conventional face masks, which improves mask efficiency and patient comfort, and also reduces the occurrence or development of pressure sores on the face of the patient over prolonged usage. In addition, the flow-inflating mask improves patient-ventilator synchrony, thereby reducing work-of-breathing and improving the successful management of patients being ventilated noninvasively.

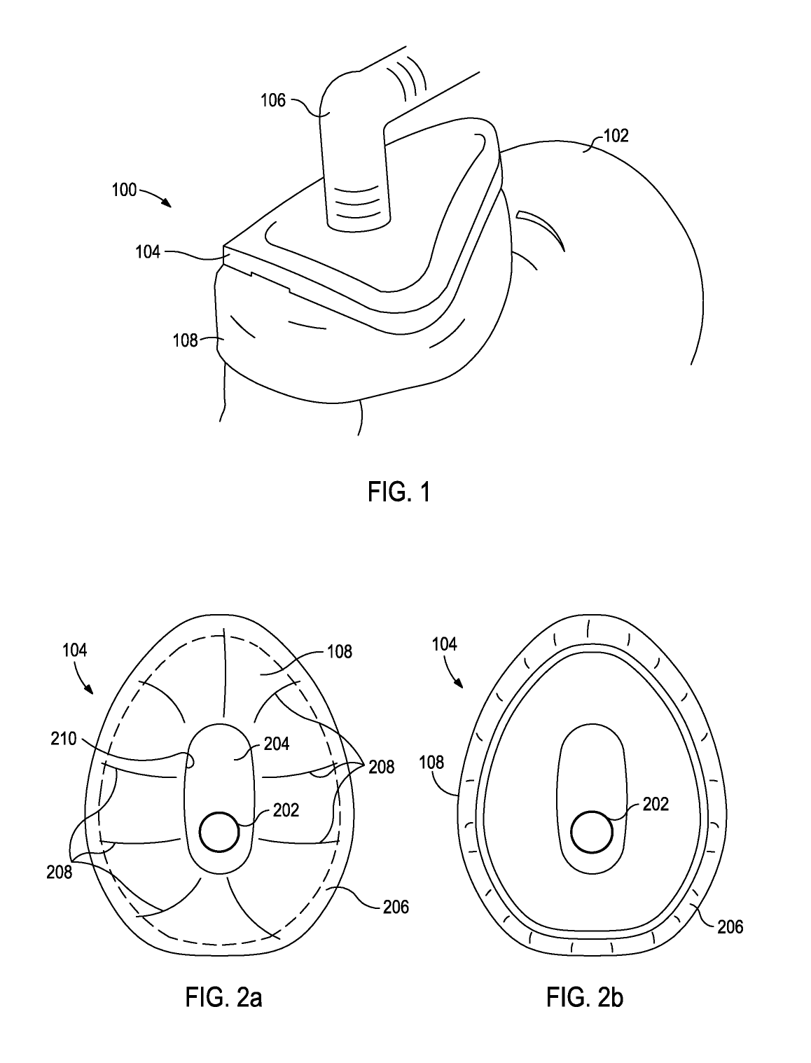

Referring to FIG. 1, illustrated is a perspective view of an exemplary flow-inflating mask 100, according to one or more embodiments disclosed. As illustrated, the mask 100 may be generally secured or otherwise seated on the face of a subject patient 102. While not illustrated, it will be appreciated that several means for securing the mask 100 to the face of the patient 102 may be used, without departing from the scope of the disclosure. In one or more embodiments, the mask 100 may be characterized as an oro-nasal mask which substantially covers both the mouth and nose of the subject patient 102. In other embodiments, however, configurations of the mask 100 may be isolated only to the nasal region or only to the mouth region, without departing from the scope of the disclosure. In yet other embodiments, the mask 100 may be designed as a full face mask that covers substantially the entirety of the face, including the eyes and substantial portions of the cheeks and forehead of the subject patient 102. Those skilled in the art will readily recognize that several alternative configurations or designs of the mask 100 are possible, but that nonetheless remain within the scope of this disclosure.

The mask 100 may include a base 104, an air supply conduit 106, and a flow-inflating skirt 108. In one or more embodiments, the base 104 may be a rigid shell made from, for example, plastic, and dimensioned to cover at least the nose and mouth of the subject patient 102. In at least one embodiment, the base 104 may be fabricated from a flexible, latex-free material, such as polyurethane. As a result, the base 104 may be a rigid but flexible shell that is soft and smooth to the touch. The flexibility of the base 104 provides greater comfort to the patient 102 due to a better, customized fit and also provides increased durability due to its ability to bend and resist breakage while forming an air pressure seal. As can be appreciated, the custom fit and durability ensure long-term leak-free use.

The air supply conduit 106 may be fluidly coupled to the base 104 and configured to communicate positive air pressure to the interior of the base 104. The air supply conduit 106 may be coupled at its other end to any type of air supply known to those skilled in the art, such as a continuous positive airway pressure machine or a noninvasive positive pressure ventilator (not shown). The air supply conduit 106 may be configured to provide any type of gaseous fluid to the mask 100 including, but not limited to, air, oxygen, helium/oxygen mixtures, nitric oxide, combinations thereof, and the like.

The flow-inflating skirt 108 is coupled to the base 104 and extends therefrom to provide a contact interface with the subject patient 102. Although the skirt 108 is generally shown in FIG. 1 as extending at least partially down the chin of the patient 102, it will be appreciated that the skirt 108 may equally seat on the top of the chin (e.g., about the base of the mouth of the patient 102), without departing from the scope of the disclosure. The skirt 108 may be coupled to the base 104 in a variety of ways including, but not limited to, mechanical fasteners, adhesives, melding techniques, combinations thereof, or the like. In at least one embodiment, the skirt 108 is sewn to the base 104 to substantially seal the skirt 108 thereto such that little to no fluid passes therethrough. The skirt 108 may be integrated or permanently fixed to the base 104 to eliminate the need for specific attachment mechanisms which would further complicate the mask 100. This also eliminates the requirement for the user or technician to install/replace a specially-designed seal mechanism, as well as the requirement for pharmacies to stock replacement seals and their associated components.

The skirt 108 may be made from one or several materials that make it compliant and thereby allow the skirt 108 to evenly interact with the patient's skin to create the necessary sealing effect. In some embodiments, the skirt 108 may be made from a generally inelastic but compliant material selected from the group consisting of cloth and fabrics, thermoplastic polymers, soft plastics, rubber, treated paper, laminated materials with synthetic or natural fibers, woven or non-woven materials, laminates of cloth and/or plastic, combinations thereof, or the like. In at least one embodiment, the skirt 108 is made from a GORE-TEX® fabric which provides an air-tight seal through the fabric, but is simultaneously soft as it contacts the skin. In alternative embodiments, the skirt 108 may be made from a cloth-like material containing elastane, polyurethane, and polyamide, thereby providing an expandable or elastic material.

Referring to FIGS. 2a and 2b, illustrated are bottom and top views, respectively, of the base 104 as coupled to the skirt 108, according to one or more embodiments. As illustrated, the base 104 may define an aperture 202 configured to accommodate the air supply conduit 106 for supplying positive pressure gases to the interior 204 of the base 104. The skirt 108 may be coupled to the base 104 at or near the outer circumference of the base 104, thereby forming a ring 206 about the base 104 that provides a substantially sealed interface between the base 104 and the skirt 108.

As depicted in FIG. 2a, the skirt 108 may include a plurality of seams 208 sewn or otherwise defined therein. In other embodiments, however, the seams 208 may be omitted and the skirt 108 may instead be made from an uncut or otherwise un-segmented section of cloth or other material that maintains the skirt 108 in a generally oval or circular shape. In some embodiments, the skirt 108 may be made from a single piece of material, such as one of the materials discussed herein. Accordingly, there may be no need to connect several pieces or sections of the skirt 108 in order to provide the entirety of the skirt 108.

The skirt 108 may define a central opening 210 which provides access for the nose and/or mouth of the patient 102 to be introduced into the interior 204 of the base 104 and thereby be in direct contact with the incoming positive pressure gases. In operation, the positive pressure gases serve to inflate the skirt 108 which reacts by billowing outwards and engaging or otherwise contacting the face of the subject patient 102. Accordingly, in at least one embodiment, the skirt 108 may be characterized as an inflatable bladder that maintains a generally oval or circular shape as it is inflated and billows outwards.

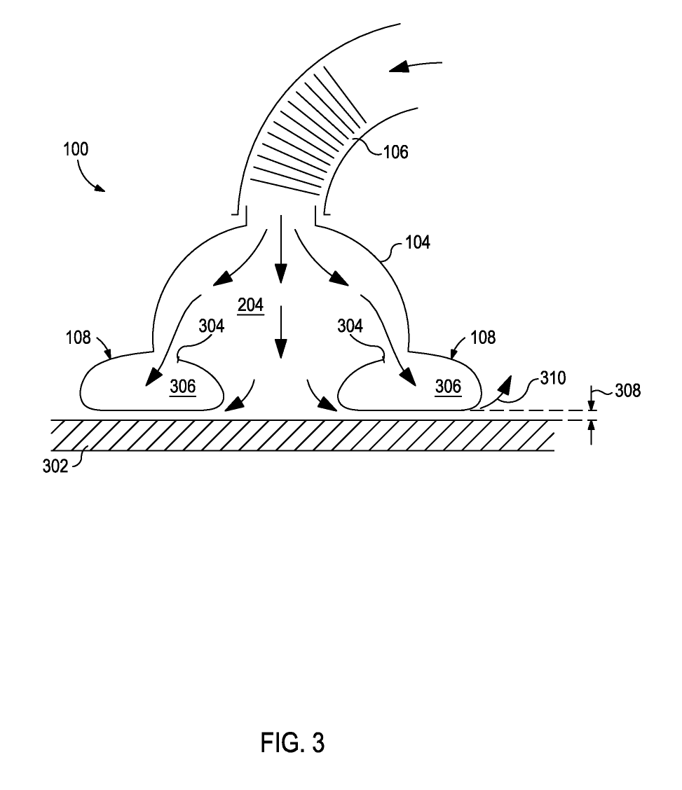

Referring now to FIG. 3, illustrated is a cross-sectional view of the flow-inflating mask 100 as it may be used during exemplary operation, according to one or more embodiments. As illustrated, the mask 100 may be in close-contact or otherwise engaged with the skin 302 of the subject patient 102 (FIG. 1). As will be appreciated, however, the mask 100 is not necessarily required to be juxtaposed directly against the skin 302, but may equally function having an interposing material arranged therebetween, such as a layer of clothing, hair, etc.

As illustrated, the skirt 108 may have a distal portion 304 that extends from the base 104 and folds or otherwise inverts at least partially towards the interior 204 of the base 104, thereby defining a cavity 306. When the continuous positive airway pressure machine or noninvasive positive pressure ventilator (not shown) is turned on, air is supplied to the air supply conduit 106 and ultimately injected into the interior 204 of the base 104. The incoming pressurized air may pass into and inflate the cavity 306, thereby resulting in a compliant and soft interface that adapts easily to the varying topography of the skin 302.

This compliant interface may prove advantageous especially in medical-use embodiments where medical tubing, such as feeding and suction tubes, are required to pass into or out of the patient's mouth and/or nose while the flow-inflating mask 100 is operational and providing positive pressure gas. Because the skirt 108 is designed as a compliant and soft structure, the cavity 306 may be able to readily deform about its circumference and accommodate medical tubings inserted between the skirt 108 and the skin 302, and the skirt 108 may nonetheless maintain its inflated status and properly seal against the skin 302.

In some embodiments, the cavity 306 may be defined about the entire inner circumference of the skirt 108, thereby forming a single annulus. In other embodiments, however, the cavity 306 may include a plurality of cavities 306 defined about the inner circumference of the skirt 108. In yet other embodiments, a flexible, inflatable, and annular tube (not shown) that defines a plurality of holes may either replace or be installed within the cavity 306. The plurality of holes may be configured to receive the pressurized air in order to inflate the annular tube and thereby inflate the skirt 108.

Function of the mask 100 may be governed by at least some of the same fluid dynamic principles that govern the function of hovercraft devices. For example, as the skirt 108 is inflated, a gap 308 may be formed or otherwise defined between the skin 302 and the skirt 108, such as between the folded back portion of the distal end 304 and the skin 302. The gap 308 may allow a portion of pressurized air 310 to leak out from the interior 204 of the base 104. As the pressurized air 310 escapes from the base 104, a Venturi effect may be generated across the gap 308 which serves to pull the skirt 108 closer to the skin 302 with an increasing suction force. As a result, the skirt 108 is pulled in close proximity to the skin 302 and floats on a thin layer of pressurized air 310 leaking through the gap 308. Consequently, the mask 100 is supported by pressurized gas and essentially floats on the face of the patient 102 as the pressurized air 310 continuously leaks out via the gap 308.

As can be appreciated, the flow-inflating skirt 108 may inflate automatically when the mask 100 receives external pressurized air via the air supply conduit 106. And because a small amount of pressurized air 310 escapes the mask 100 via the gap 308, a ventilation valve is not necessarily needed in one or more embodiments. Moreover, because of its soft and pliant material, and billowing design, the skirt 108 imposes a decreased amount of pressure on the skin 302, thereby improving mask 100 efficiency and patient 102 comfort. In addition, the mask 100 may improve patient-ventilator synchrony, thereby reducing work-of-breathing and improving the successful management of patients being ventilated noninvasively.

In some embodiments, the flow-inflating mask 100 may be used in the field of veterinary medicine. In some embodiments, the flow-inflating mask 100 may be designed and configured to extend over the entire head of the subject patient 102 so as to entirely enclose the head. In such an embodiment, the skirt 108 may be configured to self-inflate and substantially seal against, for example, the circumference of the neck of the subject patient 102. In some embodiments, the flow-inflating mask 100 may be configured to be used in conjunction with hyperbaric oxygen therapy. For example, the flow-inflating mask 100 may be designed such that it may be arranged about and seal predetermined or specific portions of the subject body in order to deliver an increased amount of oxygen to the selected portion of the body.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

The invention claimed is:

1. A flow-inflating mask for delivering a positive pressure fluid to a patient, comprising:
a rigid base dimensioned to cover at least a nose and a mouth of the patient and defining an interior configured to receive the nose and mouth; and
a flow-inflating skirt coupled to the rigid base and having a distal portion that inverts towards the interior of the base and thereby defines a cavity, the cavity being configured to receive pressurized air and inflate the flow-inflating skirt.

2. The mask of claim 1, wherein the skirt is coupled to the base by sewing the skirt to an outer circumference of the base.

3. The mask of claim 1, wherein the skirt is coupled to the base by adhesively attaching the skirt to the base.

4. The mask of claim 1, further comprising an air supply conduit coupled to the base and configured to communicate positive air pressure to an interior of the base.

5. The mask of claim 1, wherein the flow-inflating skirt is made from an inelastic material.

6. The mask of claim 1, wherein the flow-inflating skirt is made from a GORE-TEX® fabric.

7. The mask of claim 1, wherein the flow-inflating skirt is made from a cloth fabric.

8. The mask of claim 1, wherein the skirt is made from an uncut and un-segmented section of material.

9. The mask of claim 1, wherein the flow-inflating skirt defines a central opening configured to receive the nose and the mouth of the patient.

10. The mask of claim 1, wherein the flow-inflating skirt is made from a single, un-segmented section of material.

11. The mask of claim 1, wherein the cavity is defined about an entire inner circumference of the skirt.

12. A method for delivering a positive pressure fluid to a patient, comprising:
providing a flow of positive pressure fluid to a rigid face mask base defining an interior dimensioned to cover the nose and mouth of the patient; and
inflating a flow-inflating skirt with the flow of positive pressure fluid, the flow-inflating skirt being coupled to the rigid face mask base and having a distal portion that inverts towards the interior of the face mask base to define a cavity.

13. The method of claim 12, wherein inflating the flow-inflating skirt with the flow of positive pressure fluid further comprises receiving a portion of the flow of positive pressure fluid in the cavity.

14. The method of claim 12, further comprising floating the flow-inflating skirt on a layer of pressurized air escaping the face mask base via a gap defined between the flow-inflating skirt and the skin of the patient.

15. The method of claim 12, wherein the flow-inflating skirt is made from an inelastic material.

16. The method of claim 12, wherein the flow-inflating skirt is made from a GORE-TEX® fabric.

17. The method of claim 12, wherein the flow-inflating skirt is made from a cloth fabric.

18. The method of claim 12, further comprising forcing the flow-inflating skirt into proximity with the skin of the patient by ejecting a portion of pressurized air through a gap defined between the flow-inflating skirt and the skin.
